(19) United States
(12) Patent Application Publication
(10) Pub. No.: US 2009/0110758 A1
(43) Pub. Date: Apr. 30, 2009

(54) COMPOSITIONS AND METHODS FOR PROMOTING WEIGHT LOSS

(76) Inventors: John C. Seed, Princeton, NJ (US); Denise Johnson, Trenton, NJ (US)

Correspondence Address: FOX ROTHSCHILD LLP;PRINCETON PIKE CORPORATE CENTER, 2000 Market Street, Tenth Floor, Philadelphia, PA, 19103, US

(21) Appl. No.: 11/924,189

(22) Filed: Oct. 25, 2007

(51) Int. Cl. A61K 36/68; A23L 1/236; A61P 3/04; A23L 1/36

(52) U.S. Cl. 424/738; 426/548; 426/629

(57) ABSTRACT

Compositions, which include psyllium and polyethylene glycol in a weight ratio of psyllium to polyethylene glycol of from about 1:3 to about 2:1, a gum, and one or more additional ingredients selected from the group consisting of water, salt, sweeteners, flavoring agents, and preservatives are presented.

### BACKGROUND OF THE INVENTION

Today, especially in the developed industrial nations, obesity is an increasingly serious problem for the health of the population, being caused predominantly by unbalanced and excessively high-fat nutrition. The increase in the percentage of overweight people in the population is being accompanied by an increase in the consequences of obesity, which range from personal discontentment to cardiovascular disease or certain forms of diabetes. There are already a number of therapeutic procedures aimed at the treatment or prophylaxis of obesity.

Patients with morbid obesity (body mass index>29 kg/m², about 3% of the overweight population) are often encouraged to undergo bariatic surgery because, as a class, they suffer from more than four times the incidence of diabetes, cardiovascular disease, uterine and breast cancer, degenerative joint disease, and social stigmatization. Ileojejunal bypass, the first such surgery undertaken 30 years ago, has now been abandoned because of severe side effects such as poor subsequent malnutrition, fatal cirrhosis, or renal failure. Biliopancreatic by-pass, gastric by-pass, and gastric partitioning (stapling) are the current procedures, but the long term side effects have not yet been determined.

Alternatively, surgical devices have been employed to control appetite. Intragastric balloons have been placed endoscopically according to the theory that they increase the amount of gastric distension and thus augment satiety responses. However, they have been discontinued because, while they were not shown to be any better than restricted diets in promoting weight loss, their long term use was associated with severe side effects such as gastric ulceration and migration of the balloons into the small intestine resulting in intestinal obstructions.

Given the health risks associated with surgery and the implantation of surgical devices, the need exists for the development of easily ingestible compositions which promote weight loss in an individual in need thereof. Of particular interest are compositions having additional modes of utility beyond promoting weight loss in an individual.

### SUMMARY OF THE INVENTION

The present invention relates to a composition, which includes psyllium and polyethylene glycol in a weight ratio of psyllium to polyethylene glycol of from about 1:3 to about 2:1, a gum, and one or more additional ingredients selected from water, salt, sweeteners, flavoring agents, and preservatives.

Also presented is a composition for promoting weight loss, which includes an appetite-suppressing effective amount of the composition.

Another embodiment includes a method of inducing weight loss in a patient by administering the composition to a patient in need thereof.

Yet another embodiment is a frozen comestible, which includes the composition.

An additional embodiment is a drug delivery composition, which includes the composition and a pharmaceutically active compound.

### DETAILED DESCRIPTION OF THE INVENTION

The present invention provides compositions, which include psyllium and polyethylene glycol in a weight ratio of psyllium to polyethylene glycol of from about 1:3 to about 2:1, a gum, and one or more additional ingredients selected from water, salt, sweeteners, flavoring agents, and preservatives. Preferably, the composition includes psyllium and polyethylene glycol in a weight ratio of psyllium to polyethylene glycol of 2:1, a gum, water, salt, acesulfame K, sucralose, potassium sorbate, calcium propionate, and a flavoring agent.

Exemplary gums include, but are not limited to, kappa carrageenan, lambda carrageenan, iota carrageenan (e.g. Gelcarin), locust bean, and xanthan.

The amount of gum is from about 0.02 weight % to about 2.0 weight % of the product, preferably about 0.2 weight % to about 1.0 weight %.

When present, the amount of total sweetener is from about 2.0 weight % to about 8.0 weight % of the product, preferably about 2.4 weight % to about 4.0 weight %. Suitable sweeteners for use in the present invention include sugar sweeteners, sugarless sweeteners, and artificial sweeteners (e.g. high intensity artificial sweeteners). Sugar sweeteners generally include saccharide-containing components commonly known in art, including but not limited to, sucrose, dextrose, maltose, dextrin, dried invert sugar, fructose, galactose, corn syrup solids, and the like, alone or in combination. Sugarless sweeteners include, but are not limited to, sugar alcohols such as sorbitol, mannitol, xylitol, hydrogenated starch hydrolysates, maltitol, and the like, alone or in combination.

Artificial sweeteners include, but are not limited to, sucralose, aspartame, N-substituted APM derivatives such as neotame, salts of acesulfame (e.g. acesulfame K), alitame, saccharin and its salts, cyclamic acid and its salts, glycyrrhizin, dihydrochalcones, thaumatin, monellin, and the like, alone or in combination. Preferably, the sweetener is an artificial sweetener.

When present, the amount of total flavoring agent is from about 0.3 weight % to about 20 weight % of the product, preferably about 3.0 weight % to about 10 weight %. Examples of flavoring agents include conventional flavoring agents such as cocoa, chocolate, vanilla, fruit (e.g. peach, fruit punch, cranberry, strawberry, raspberry, and citrus), milk (e.g. powdered or condensed), kahlua, coffee, mocha, liqueur flavor, garlic, onion, scallion, pepper, cilantro, celery, basil, oregano, mustard, mint, parsley, thyme, rosemary, fennel, ginger, mushroom, cinnamon, soy, vinegar, tea, radish, prosciutto, anchovy, combinations thereof, and the like. Preferred flavoring agents include cocoa, fruit, and milk. The flavoring agent(s) can be used in any form suitable for the final composition. Preferred forms include powders and syrups. In one embodiment, the composition is flavored using a dried fruit or vegetable powder. For example, a cranberry powder is prepared by soaking cranberries to extract natural sugars. The cranberries are then dried and ground into a fine powder, which is blended into the composition.

When present, the amount of total preservative is from about 0.1 weight % to about 2.0 weight % of the product, preferably about 0.4 weight % to about 1 weight %. Preservatives can be added to the composition to prolong the salability and/or edibility of the composition. For example, preservatives can be added to inhibit the growth of fungi and/or bacteria. Exemplary preservatives include potassium sorbate and calcium propionate.

Optionally, the composition further includes a colorant and/or a whitener (e.g. titanium dioxide).

Optionally, the psyllium is heated to provide a final composition having a softer texture compared with a final composition in which the psyllium was not heated.

In one embodiment, a composition for promoting weight loss includes an appetite-suppressing effective amount of a composition of the present invention. Optionally, the composition further includes pyruvate, sitagliptin, or a combination thereof. The composition can be administered to effectively suppress, inhibit, reduce or otherwise curtail an appetite in an individual. The appetite-suppressing compositions therefore can also be administered to an individual in order to control weight or to treat obesity. As used herein, “appetite-suppressing” refers to a statistically significant and detectable or measurable reduction in food intake (over a time period of at least about 24 hours) when food is available on an ad libitum, or an equivalent schedule. Preferably, the appetite-suppressing effective amount administered to the individual ranges from about 1 teaspoon to about 5 tablespoons. The composition for promoting weight loss can be packaged in multiples of the appetite-suppressing effective amount from which individual portions can be obtained. The appetite-suppressing effective amount is preferably administered to the individual prior to a meal or in place of a snack between meals.

In another embodiment, a drug delivery composition includes the composition of the present invention and a pharmaceutically active compound. Bitterness inhibitors or taste maskers can also be added to the drug delivery composition to give a reduced unpleasant taste.

Yet another embodiment includes a frozen comestible, which includes a composition of the present invention. The frozen comestible can have any suitable form, such as, for example, a popsicle form.

The following non-limiting examples set forth hereinbelow illustrate certain aspects of the invention.

### EXAMPLES

TABLE I
Composition I

| Ingredient | Amount | |
|---|---|---|
| Psyllium (heated) | 80 | grams |
| Polyethylene glycol | 40 | grams |
| Colloid 710H | 1.5 | grams |
| Acesulfame K | 10.5 | grams |
| Sucralose | 10 | grams |
| Potassium sorbate | 1.5 | grams |
| Calcium propionate | 2 | grams |
| Strawberry powder | 2 | grams |
| Concentrated salt solution | 2 | mL |
| Water | 700 | mL |

An obese 52-year old male patient was interested in losing weight. The patient was placed on Composition I and instructed to consume 4 to 5 tablespoons of the composition 15 to 20 minutes before each meal. His weight loss progress is set forth in Table II:

TABLE II
Weight loss

| | Number of Days on |
|---|---|
| Weight (lbs) | Composition I |
| 286.25 | 0 |
| 282.50 | 4 |
| 288.75 | 8 |
| 282.25 | 11 |
| 281.75 | 15 |
| 280.75 | 17 |
| 278.00 | 24 |
| 277.75 | 31 |
| 273.50 | 38 |
| 268.00 | 45 |
| 266.25 | 55 |
| 269.75 | 62 |
| 262.75 | 69 |
| 261.00 | 76 |
| 259.25 | 81 |
| 257.00 | 90 |
| 256.25 | 97 |
| 256.00 | 105 |
| 252.00 | 125 |

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims.

1. A composition comprising psyllium and polyethylene glycol in a weight ratio of psyllium to polyethylene glycol of from about 1:3 to about 2:1, from about 0.02 wt % to about 2.0 wt % of the total composition of a gum, and one or more additional ingredients selected from the group consisting of sweeteners in an amount from about 2.0 wt % to about 8.0 wt % of the total composition, flavoring agents in an amount from about 0.3 wt % to about 20 wt % of the total composition, and preservatives in an amount from about 0.1 wt % to about 2.0 wt % of the total composition.

2. The composition of claim 1, wherein said sweeteners are artificial sweeteners.

3. The composition of claim 1, wherein said preservatives are selected from the group consisting of potassium sorbate and calcium propionate.

4. The composition of claim 1 comprising psyllium and polyethylene glycol in a weight ratio of psyllium to polyethylene glycol of 2:1, a gum, water, salt, acesulfame K, sucralose, potassium sorbate, calcium propionate, and a flavoring agent.

5. A composition for promoting weight loss comprising an appetite-suppressing effective amount of the composition of claim 1.

6. A composition for promoting weight loss comprising an appetite-suppressing effective amount of the composition of claim 4.

7. A method of inducing weight loss in a patient comprising administering the composition of claim 1 to a patient in need thereof.

8. The method of claim 7, wherein the composition further comprises pyruvate, sitagliptin, or a combination thereof.

9. A method of inducing weight loss in a patient comprising administering the composition of claim 4 to a patient in need thereof.

10. A frozen comestible comprising the composition of claim 1.

11. A frozen comestible comprising the composition of claim 4.

12. A drug delivery composition comprising the composition of claim 1 and a pharmaceutically active compound.

13. A drug delivery composition comprising the composition of claim 4 and a pharmaceutically active compound.
